(19) United States
(12) Patent Application Publication
(10) Pub. No.: US 2005/0084456 A1
(43) Pub. Date: Apr. 21, 2005

(54) Functionalized particles

(76) Inventors: Liping Tang, Arlington, TX (US); Sheng Zhang, Arlington, TX (US); Hong Weng, Arlington, TX (US)

Correspondence Address: Monique A. Vander Molen;Gardere Wynne Sewell LLP, 3000 Thanksgiving Tower, Suite 3000, 1601 Elm Street, Dallas, TX, 75201-4767, US

(21) Appl. No.: 10/690,466

(22) Filed: Oct. 21, 2003

(51) Int. Cl. A61L 9/04; A61K 9/14; C12N 15/87

(52) U.S. Cl. 424/46; 435/459

(57) ABSTRACT

The present invention provides functionalized particles and methods for delivering said particles capable of crossing a physiologic barrier and exerting an effect. In one embodiment, the present invention provides a method of delivering a particle to a mammal comprising the steps of contacting a functionalized particle with a tag and introducing the functionalized and tagged particle to a mammal, wherein the functionalized portion of the particle is selected from the group consisting of acrylic acid, 2-hydroxyethyl acrylate, 2-acrylamido-2-methyl-1-propanesulfonic acid, allylamine, carboxyl group, hydroxyl group, sulfonic group, aldehyde and amine group. The particle is a biodegradable or nodegradable polymer and less than 1.0 mm in diameter.

### STATEMENT REGARDING FEDERALLY SPONSORED RESEARCH OR DEVELOPMENT

The present application was supported in part by the National Institutes of Health grant number EB-00287. The U.S. government may therefore have certain rights in the invention.

### BACKGROUND OF THE INVENTION

The present invention relates to the general field of clinically and therapeutically effective agents for the prevention and treatment of diseases, and more particularly, to methods and a delivery system for targeting agents and diagnostics to specific tissues and/or organs.

As discussed herein, the diagnosis and delivery of pharmaceutical or diagnostic compositions may be administered in one of several forms, generally including injection, by mouth (oral), and/or by implantation. Upon delivery of any therapeutic or prophylactic agent into the body, the agent next encounters several physiologic barriers preventing its direct delivery to a specific organ or tissue. The presence of these physiological barriers to drug delivery to a specific organ/tissue substantially limits efficacy of the agent when used to treat or prevent a disease or for diagnostic purposes. For example, to adhere or penetrate into most cells, a therapeutic/prophylactic agent often has to pass through blood vessel walls (e.g., endothelial cell junction), interstitial space, and cell membranes. Penetration of a therapeutic/prophylactic agent through these physiological barriers is poor especially for the most promising synthetic and natural drugs as well as macromolecular therapeutic agents such as chemotherapy drugs, monoclonal antibodies, cytokines, antisense oligonucleotides, and gene-targeting vectors

As an example, drug delivery to the eye generally takes on one of two forms, eye drops and implants. Eye drops have been the accepted method and with drops, active ingredients are mixed with liquids and applied to the surface or anterior portion of the eye. Unfortunately, it is not uncommon for as little as 1% or less of the active ingredients in the drops to reach the posterior portion of the eye. This is in part because agents administered to the anterior portion of the eye do not effectively cross the blood-eye barrier. However, it is the posterior portion of the eye that is generally associated with most ocular diseases and other conditions that lead to visual impairment or loss of vision, such as ocular, uveal, retinal and retinochoriodal diseases or melanomas. With such low level of drug delivered to the eye, especially the posterior portion of the eye, most drugs fail to effectively and rapidly cure the condition. Hence, prolonged treatments (6 months or longer) is often required. As an alternative, devices, such as implants have been developed. This invasive procedure requires surgical intervention and is associated with a higher risk of side effects than other forms of pharmaceutical administration. Therefore, neither drops nor implants are a safe and effective means of delivery of pharmaceutical or diagnostic compositions to the eye.

Drugs that are administered to parts of the body other than the eye are generally able to circulate in the blood; however, most ocular drugs cannot cross the blood-brain barrier. It is generally believed that capillaries (e.g., simple tubes of endothelial cells) surround the brain and its nervous network and prevent the transmigration of such drugs (i.e., those in chemical, peptide, protein, virus or cellular forms). Where there is minimal penetration across the blood-brain barrier, extremely high doses and multiple injections of a drug are needed to achieve their therapeutic effect. Unfortunately, increasing the dose of any drug is generally associated with an increased risk of adverse events and tissue toxicity. In addition, those drugs that cannot cross the blood-brain barrier, can not be used to treat diseases of the eye, ear, brain, or central nervous system.

Therefore, there remains a particular need for a drug delivery system that works in a targeted manner, may pass physiologic barriers and reduce the potential for detrimental side-effects or tissue toxicity.

### SUMMARY OF THE INVENTION

To solve the current problem of safely and effectively delivering preventative, diagnostic, or therapeutic compositions to a specific tissue and/or organ, especially across a physiologic barrier, the present invention combines targeted pharmaceutical compositions with technologic advances in micro- and nanoparticle preparation to safely, selectively, and effectively deliver an agent and/or diagnostic to a specific tissue and/or organ (e.g., eye, brain, nerves, pancreas, kidney).

Generally and in one form, the present invention provides methods for delivering preventative, diagnostic, or therapeutic micro- and nanoparticle compositions (e.g., functionalized particles) to one or more specific locations within a body, such as an organ or tissue, especially targets that are inaccessible due to a physiologic barrier.

The present invention additionally provides for a method of delivering a particle to a mammal comprising the steps of contacting a functionalized particle with a tag; and introducing the functionalized and tagged particle to a mammal, wherein the functionalized portion of the particle is selected from the group consisting of acrylic acid, 2-hydroxyethyl acrylate, 2-acrylamido-2-methyl-1-propanesulfonic acid, allylamine, carboxyl group, hydroxyl group, sulfonic group, aldehyde group, and amine group, and wherein the particle is a biodegradable or nodegradable polymer and less than 10 mm in diameter. The functionalized particle is a polymer selected from the group consisting of polyelectrolyte, hydroxypropyl cellulose, N-isopropylacrylamide, and hyaluronan. The tag is selected from the group consisting of drug, antibodiy, ligand, antigen, protein, peptide, nucleic acid sequence, fatty acid moiety, carbohydrate moiety, label, light-emitting species, radioactive species, nuclear species, and combinations thereof. The particle is, thus, protective or therapeutic for one or more diseases of the eye, liver, brain, pancreas, spleen, kideny, or lung.

In another form, the present invention provides functionalized particles for prevention, treatment and diagnosis of organ/tissue diseases and/or conditions. The functionalized particles of the present invention are useful for delivering drugs to a target and may be controlled for size and/or for the particular target. In one embodiment, the functionalized particle is additionally modified, such as by additional of a chemical, biologic component (antibody, nucleic acid, amino acit, fatty acid, etc.), or labeled for detection.

Another aspect of the present invention provides methods for delivering functionalized particles (e.g., drug-particles and diagnostics) across physiologic barriers (e.g., blood-eye barrier, blood-brain barrier, blood-cell barrier, endothelial cell junction barrier). Drugs or diagnostic agents are incorporated into functionalized particles of the present invention particles that have been fabricated as described herein. These nanoparticles may be further coated with an appropriate substance (e.g., surfactant, targeted compound, chemical, nucleic acid sequence, amino acid sequence, antibody, antigen) and given to animals or humans.

The nanoparticles of the present invention achieve one or more of the following benefits: (1) reduce the delivery dose required for a therapeutic drug or diagnostic agent while maintaining the biologic or diagnostic potency at its target; (2) allows drugs that normally do not cross the biologic barriers to penetrate; (3) provide a drug with the ability to concentrate at a target; and (4) reduce peripheral side effects after drug administration. The biologic composition may be designed to target one or more specific components of a tissue or organ, such as a cell surface antigen.

The methods of the present invention are powerful tools to effectively deliver drugs with preventative, therapeutic, or diagnostic properites to inaccessible areas of the body, such as the brain or eye. Custom designed products of the present invention may be used for therapeutic, diagnostic, technologic, research and development applications. These and other objects, embodiments and features of the present invention will be apparent from the detailed description and the drawings.

### BRIEF DESCRIPTION OF THE SEVERAL VIEWS OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 depicts a schematic in accordance with one aspect of the present invention;

FIG. 2 depicts a simplified strategy to target the delivery of a nanoparticle in accordance with one aspect of the present invention;

FIG. 3 depicts the presence of nanoparticles in uveal tissue (A) 12 hours after intravenous administration of FITC-labeled NIPA nanoparticles (˜100-500 nm) and (B) after intravenous administration of FITC alone, wherein there is no accumulation in uveal tissue;

FIG. 4 depicts the penetration and accumulation of nanoparticles in uveal tissue seven days after intravenous administration of FITC-labeled NIPA-amine nanoparticles (˜100 nm) as viewed by (A) fluorescent microscopy and (B) light microscopy after H&E staining;

FIG. 5 depicts the penetration and accumulation of nanoparticles in the brain stem seven days after intravenous administration of FITC-labeled NIPA-amine nanoparticles (˜100 nm) as viewed by (A) fluorescent microscopy and (B) light microscopy after H&E staining;

FIG. 6 depicts the penetration and aggregation of nanoparticles in the lung tissue six days after intravenous administration of FITC-labeled NIPA-amine nanoparticles (˜100 nm) observed with (A) fluorescent microscopy and (B) light microscopy after H&E staining;

FIG. 7 depicts the penetration and buildup of nanoparticles throughout the liver tissue four days after intravenous administration of FITC-labeled NIPA-amine nanoparticles (˜100 nm) as viewed by (A) fluorescent microscopy and (B) light microscopy after H&E staining;

FIG. 8 depicts the penetration and accumulation of nanoparticles throughout the pancreas tissue four days after intravenous administration of FITC-labeled NIPA-amine nanoparticles (˜100 nm) as viewed by (A) fluorescent microscopy and (B) light microscopy after H&E staining;

FIG. 9 depicts the distribution and accumulation nanoparticles throughout the kidney tissue four days after intravenous administration of FITC-labeled NIPA-amine nanoparticles (˜100 nm) as viewed by (A) fluorescent microscopy and (B) light microscopy after H&E staining;

FIG. 10 depicts the distribution and accumulation of nanoparticles throughout the spleen tissue four days after intravenous administration of FITC-labeled NIPA-amine nanoparticles (˜100 nm) as viewed by (A) fluorescent microscopy and (B) light microscopy after H&E staining;

FIG. 11 shows that of NIPA-amine nanoparticles (˜100 nm) do not (B) trigger adverse responses in retinal tissue following intravitreal injection as compared to control tissue (A) nor (D) result in inflammatory responses following implantation as compared to the control tissue (C);

FIG. 12 depicts results after intravitreal injection of NIPA-amine microparticles of 50 μm (A and B) and non-modified NIPA nanoparticles of 100 nm (C and D) as viewed by (A and C) fluorescent microscopy and (B and D) electron microscopy; and

FIG. 13 depicts the distribution of NIPA-amine nanoparticles of 100 nm (A) three hours following implantation, (B) three and a half hours following implantation with penetration into the retina, (C) and as viewed with an electron microscope.

### DETAILED DESCRIPTION OF THE INVENTION

Although making and using various embodiments of the present invention are discussed in detail below, it should be appreciated that the present invention provides many inventive concepts that may be embodied in a wide variety of contexts. The specific aspects and embodiments discussed herein are merely illustrative of ways to make and use the invention, and do not limit the scope of the invention.

To facilitate the understanding of this invention, a number of terms are defined within. Terms defined and used herein have meanings as commonly understood by a person of ordinary skill in the areas relevant to the present invention. The terminology and examples herein are used to describe specific embodiments of the invention, but their usage does not limit the invention, except as outlined in the claims.

As used herein, terms such as “drug,” “agent,” “pharmaceutical drug” or “pharmaceutical composition” may be used interchangeably. In general, these terms refer to any chemical substance used in the treatment, cure, prevention, or diagnosis of a disease or condition or to otherwise change the physical or mental status of a human or other animal, regardless of molecular weight. A pharmaceutical composition may also be prepared using a drug in combination with a drug delivery vehicle of the invention. The pharmaceutical composition can comprise a drug in a suitable polymeric form and a biologically acceptable carrier. Suitable polymeric forms include microcapsules, microparticles, films, polymeric coatings, and nanoparticles.

As used herein, terms such as “microparticle,” “nanoparticle,” “microscopic particle” or “functionalized particle” are used to refer to microscopic (few micrometers in size to few millimeters in size) or submicroscopic (less than one micrometer) solid colloidal objects, generally cylindrical or spherical in shape with a semipermeable shell or shaped like a permeable nano-ball. One or more drugs or other relevant materials (e.g., those used for diagnostic purposes, such as in nuclear medicine or in radiation therapy) may be dissolved within the nanoparticles, entrapped, encapsulated, absorbed, adsorbed, covalently linked, or otherwise attached. Furthermore, particles of the present invention may be coated. When a relevant material as just described is added to a particles, it may be considered a tagged particle.

The particle of the present invention is generally made as a metal particle, carbon particle, graphite particle, polymer particle, hydrogel particle, liquid particle or porous particle. Thus, micro- and nanoparticles may be metal, carbon, graphite, polymer, and may be loaded with a light or color absorbing dye, an isotope, a radioactive species, or be porous having gas-filled pores. As used herein, the term “hydrogel” refers to a solution of polymers, sometimes referred to as a sol, converted into gel state by small ions or polymers of the opposite charge or by chemical crosslinking.

Suitable polymers and polyelectrolytes of the present invention include copolymers of water soluble polymers, including, but not limited to, dextran, derivatives of poly-methacrylamide, PEG, maleic acid, malic acid, and maleic acid anhydride and may include these polymers and a suitable coupling agent, including 1-ethyl-3(3-dimethylaminopropyl)-carbodiimide, also referred to as carbodiimide. Polymers may be degradable or nondegradable in the body or polyelectrolyte materials. Degradable polymer materials include poly-L-glycolic acid (PLGA), poly-DL-glycolic, poly-L-lactic acid (PLLA), PLLA-PLGA copolymers, poly(DL-lactide)-block-methoxy polyethylene glycol, polycaprolacton, poly(caprolacton)-block-methoxy polyethylene glycol (PCL-MePEG), poly(DL-lactide-co-caprolactone)-block-methoxy polyethylene glycol (PDLLACL-MePEG), some polysaccharide (e.g., hyaluronic acid, polyglycan, chitoson), proteins (e.g., fibrinogen, albumin, collagen, extracellular matrix), peptides (e.g., RGD, polyhistidine), nucleic acids (e.g., RNA, DNA, single or double stranded), viruses, bacteria, cells and cell fragments, as examples. Nondegradable materials include natural or synthetic polymeric materials (e.g., polystyrene, polypropylene, polyethylene teraphthalate, polyether urethane, polyvinyl chloride, silica, polydimethyl siloxane, acrylates, arcylamides, poly (vinylpyridine), polyacroleine, polyglutaraldehyde), some polysaccharides (e.g., hydroxypropyl cellulose, cellulose derivatives, dextran®, dextrose, sucrose, ficoll®, percoll®, arabinogalactan, starch), and hydrogels (e.g., polyethylene glycol, ethylene vinyl acetate, N-isopropylacrylamide, polyamine, polyethyleneimine, poly-aluminum chloride).

Should coating of the particle be required, typical materials suitable for coating of the particles of the present invention may include, as an example surfactants such as those including fatty acid esters of glycerols, sorbitol and other multifunctional alcohols (e.g., glycerol monostearate, sorbitan monolaurate, sorbitan monoleate), polysorbates, poloxamers, poloxamines, polyoxyethylene ethers and polyoxyethylene esters, ethoxylated triglycerides, ethoxylated phenols and ethoxylated diphenols, surfactants of the Genapol TM and Bauki series, metal salts of fatty acids, metal salts of fatty alcohol sulfates, sodium lauryl sulfate, and metal salts of sulfosuccinates.

The particles of the present invention are produced by conventional methods known to those of ordinary skill in the art. Techniques include emulsion polymerization in a continuous aqueous phase, emulsion polymerization in continuous organic phase, interfacial polymerization, solvent deposition, solvent evaporation, dissolvation of an organic polymer solution, cross-linking of water-soluble polymers in emulsion, dissolvation of macromolecules, and carbohydrate cross-linking. These fabrication methods can be performed with a wide range of polymer materials mentioned above. Examples of materials and fabrication methods for making nanoparticles have been published. (See Kreuter, J. 1991. Nanoparticles-preparation and applications. In: M. Donbrow (Ed.): Microcapsules and nanoparticles in medicine and pharmacy. CRC Press, Boca Raton, Fla., pp. 125-148; Hu, Z, Gao J. Optical properties of N-isopropylacrylamide microgel spheres in water. Langmuir 2002;18:1306-67; Ghezzo E, et al., Hyaluronic acid derivative microspheres as NGF delivery devices: Preparation methods and in vitro release characterization. Int J Pharm 1992;87:21-29; incorporated by reference herein.) The drug or a diagnostic agent can either be adsorbed or absorbed to a premade nanoparticle or it can be incorporated into the nanoparticle during the manufacturing process. Methods of absorption, adsorption, and incorporation are common knowledge to those skilled in the art. The choice of the monomer and/or polymer, the solvent, the emulsifier, the coating and other auxiliary substances will be dictated by the particular nanoparticle being fabricated and can be chosen, without limitation and difficulty, by those skilled in the art. The ratio of drug to particle (e.g., polymer) may be varied as appropriate for drug delivery. In addition, the removal of solvent or emulsifier may include a number of methods well known to one of ordinary skill in the art.

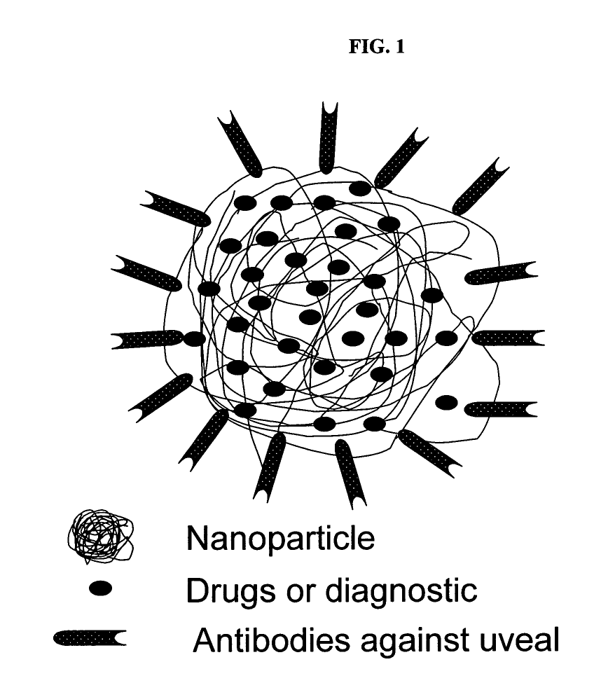

FIG. 1 is a schematic of “smart” functionalized particles of the present invention that deliver one or more specific drugs to one or more specific targets (e.g., cell, tissue, organ). The “smart” nanopaticle diameter is generally <1.0 micrometer and made of a material that is biodegradable or non-degradable (e.g., polymer, metal). On the nanoparticle, there is a “tag.” For treatment and prevention of diseases, the tag is preferably a drug that contacts the particle, either by coating to the surface, conjugating, or blending during nanoparticle formation, as examples. To target the “smart” particle to a specific sight there may also be a modification of the particle with one or more specific antibodies, antigens, peptides, or other molecular ligands. The molecular ligand is often provided as covalent modification to the outer surfaces of the functionalized particle.

As used herein, the terms “tagging” or “tag” include the addition of a material or molecule with an ability to modify the particle. Such tags may be drugs or may be molecular ligands (e.g., molecules/compounds) that recognize the cell, organ or tissue of interest, such as antibodies, antigens, proteins, peptides, nucleic acid sequences, fatty acid or carbohydrate moieties, as examples. They may also be modified compounds or polymers that mimic recognition sites on cells, organs, or tissues. The tags may recognize a portion of the tissue or organ, including but not limited to a cell surface marker, cell surface receptor, immune complex, antibody, MHC, extracellular matrix protein, plasma, cell membrane, extracellular protein, cofactor, growth factor, fatty acid, lipid, carbohydrate chain, gene sequence, or cytokine. Examples of cell surface markers are gp100, melan-A, melanin, ICAM-1, decay-accelerating factors, membrane metallo-endopeptidase (neutral endopeptidase, enkephalinase, lymphoblastic leukemia antigen, CD10, as examples), CD1, CD34, CD200, CD95, lymphocyte markers (CD3, CD8, CD4, CD5, CD69, S-antigen, as examples), intermediate filament protein (e.g., vimentin and keratin), E selectin, P selectin, other cell signals (e.g., Fas). Examples of some other proteins or cytokines that may be recognized by one or more tags include collagen, beta B1-crystallin, elastin, fibrin, fibronectin, fibrinogen, homocysteine, hyaluronin, melanin, myelin basic protein, retinoid-binding proteins, tumor necrosis factor, interleukin-1, interleukin-2, interleukin-6, macrophage migration inhibitory factor, interleukin-8, endothelin, lipoteichoic acid, complement components, interferon, transforming growth factor, leukotrienes, leukotriene receptors, as examples. The tag may also consist of label, such as a light-emitting, isotopic, nuclear or radioactive species or image contrast agent as used for diagnostic purposes. In another embodiment, the tag is a combination of one or more of the above-referenced tags contacting a larger molecule.

Drugs suitable for use with the present invention drugs include contrast agents (intravenous, intravascular, tumor-specific, hepatobiliary, reticuloendothelial, as examples), steroids, non-steroidal anti-inflammatory drugs, chemotherapy drugs, disease-specific drugs such as ocular drugs, neurologic agents, histamine-blockers, antiinfectives, including antibiotics, antifungals, antivirals, antiparasitics, antimalarials, chemotherapeutic agents, antiinflammatories, those acting a cellular or synaptic junctions, general and local analgesics and anesthetics, hypnotics and sedatives, drugs for the treatment of psychiatric disorders, protective agents, immunosuppressives; hormones and hormone antagonists; heavy metals and heavy metal antagonists; antagonists for non-metallic toxic agents, antispasmodics, antihistamines, antinauseants, relaxants, stimulants, cerebral dilators, psychotropics, anti-manics, vascular dilators and constrictors, anti-hypertensives, migraine treatments, hyper- or hypo-glycemic agents, mineral or nutritional agents, anti-obesity drugs, anabolics and anti-asthmatics, as examples. The drugs also include peptides, proteins, “sense” and “anti-sense” oligonucleotides, viral and non-viral gene therapy products, agents such as transmitters and their respective receptor-agonists and -antagonists, their respective precursors or metabolites. As such, there is no limitation on the drug or drug ingredient(s) that may be used with the present invention.

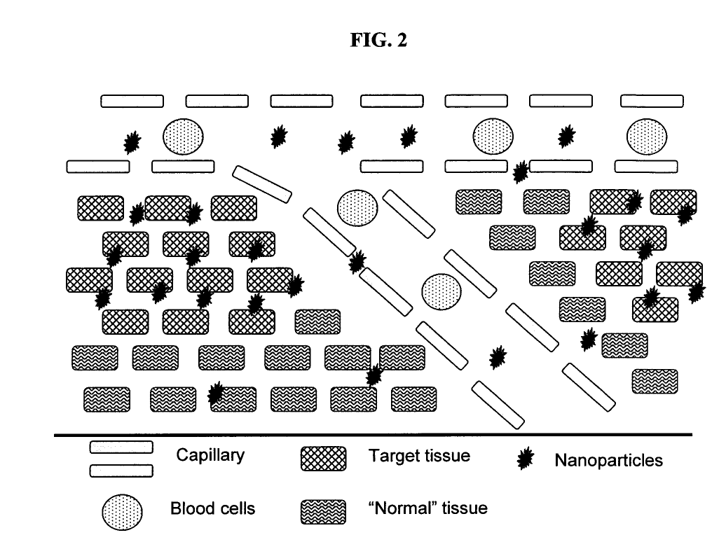

FIG. 2 is a schematic depicting the targeting strategy of the present invention. Targeted delivery of nanoparticles of the present invention occur because functionalized nanoparticles of the present invention are able to cross physiologic barriers, such as capillaries, and penetrate as well as accumulate into tissue. Microparticles do not exhibit such properties. The nanoparticles provided by the present invention also include specific “tags.” With nanoparticle “tags,” the nanoparticles will accumulate only in targeted tissue that recognize the tag. There are minimal systemic complications with these functionalized and tagged particles, because the particles do not accumulate where there is no recognition of the tag. With the present invention, disease treatment, tissue repair and/or cell/material removal may occur with reduced side effects, because only the diseased or injured tissue is targeted.

Taking ocular disease as an example, the extent of the disease is worldwide; greater than 150 million people worldwide are found to have a visual disability and in need of treatment. It is estimated that 38 million persons are living with blindness with an additional 110 million people exhibiting low vision and at risk of becoming blind. Most diseases and conditions of the eye, especially those that may lead to blindness are located on the posterior portion of the eye. While current treatments for posterior conditions and diseases are through addition of one or more drugs to the front or anterior portion of the eye, this method does not have a large affect on the posterior portion, especially in uveal disease (e.g. uveitis, uveal dystrophy, choroidal dystrophies), retinal disease (e.g. macular dystrophies, macular disorders, congenital or hereditary diseases or retinal dystophies, vascular retinopathy, trauma retinopathy, diabetic retinopathy, hypertensive retinopathy and systemic retinopathy), ocular tumors (e.g. retinoblastoma, uveal melanoma, metastatic tumors) and scleral disease (e.g. sclerititis). The composition and method of the present invention is specifically designed to treat such conditions, as examples. Other tissue that may be treated by the present invention include those that reside in an organ such as breast, lung, digestive tract, heart, spleen, blood, bone, skin, brain, liver, skin, kidney, GI organ, prostate, bladder and gynecologic organ, as examples.

The versatility of the drug-delivery system of the present invention is that the particle of the present invention may be used in combination with other techniques that may further improve its delivery, such as ultrasound, radiation, microwaves, magnetic fields, electric stimulation, or the introduction of one or more additional drugs.

Techniques for making and targeting particles of the present invention are further described by illustration below.

Synthesis of Hydroxypropyl Cellulose (HPC) Nanoparticles.

In one embodiment, HPC nanoparticles are synthesized by chemically crosslinking collapsed HPC polymer chains in salt water without any surfactant above the lower critical solution temperature (LCST) (at least about 41 degrees Centigrade). Methods include modifications from published method. (See Gehrke S H, Synthesis, Equilibrium Swelling, Kinetics Permeability and Applications of Environmentally Responsive Gels. Adv Polym Sci. 1993;110: 81; Lu XH, Hu ZB, Gao J, Synthesis and Light Scattering Study of Hydroxypropyl Cellulose Microgels. Macromolecules. 2000;33: 8698-702; incorporation by reference herein.) The size distributions of HPC nanoparticles may change by varying surfactant concentration, polymer concentrations, crosslinker densities, and reaction temperatures, as is known to one of ordinary skill in the art.

Synthesis of N-isopropylacrylamide (NIPA) Nanparticles.

N-isopropylacrylamide (NIPA) nanoparticles are synthesized following disclosed methods with specific modifications. Different building blocks of NIPA-derivative nanoparticles, with various particle sizes and crosslinker densities, are synthesized using an emulsion polymerization method. (See Pelton R H, Chibante P, Preparation of Aqueous Latices with N-Isopropylacrylamide. Colloids and Surfaces. 1986;20: 247-56; incorporated herein by reference.)

Nanoparticle examples of the present invention include NIPA co-polymerized with acrylic acid (AA), NIPA with 2-hydroxyethyl acrylate (HEAc), NIPA with HEAc and 2-acrylamido-2-methyl-1-propanesulfonic acid (AAMPSA) and NIPA with allylamine. The NIPA has thermally responsive properties; the AA, the HEAc, the AAMPSA, and the allylamine provide aldehyde, carboxyl (—COOH), hydroxyl (—OH), sulfonic (—SO₃⁻), and amine (NH₃) groups, respectively, for binding biomolecules (e.g., molecular ligands), drugs or other tags.

Synthesis of Hyaluronan (HA) Derivative Nanoparticles.

Because of its biological origin and biodegradable properties, HA is a great molecule for synthesizing as a drug delivery device. HA nanoparticles are synthesized using modified procedures. (See Ghezzo E, et al., Hyaluronan derivative microspheres as NGF delivery devices: Preparation methods and in vitro release characterization. Int J Pharm. 1992;87: 21-9; incorporation by reference, herein.) For the present invention, an oil-water emulsion is prepared in the internal phase (as at least about 6% HA) and the external phase is a mineral oil containing different amounts of surfactant (e.g., Arlacel®). Following mixing and stirring, ethyl acetate, the extraction solvent, is added to the emulsion (at least about 2:1 v/v) to form HA particles.

Correlating Nanoparticle Structures with Chemical Reactions and Chemical Compositions.

The size distribution of HPC, NIPA, HA and other nanoparticles are measured by light scattering as a function of chemical reaction time, ultrasound power, initial monomer concentrations, and initial crosslinker concentrations. HPC, NIPA, and HA nanoparticles may be readily modified and tagged during synthesis as is well known by one of ordinary skill. In addition, particles from materials previously described may be made into functionalized particles of the present invention using known techniques.

The Production of Covalently Coated and Tagged Nanoparticles.

As an example, the treatment of a uveal melanoma, is described to illustrate the utility of the present invention to specifically target a tissue and/or organ. Three nanoparticles with high affinity to uveal melanoma were used. IgG monoclonal antibodies included: HMB-45 and NKI/beteB, both raised against gp100 (Dako Corp. and Lab Vision Corp, respectively) and A103 and M2-9E3, raised against melan-A ( Novus Biologials Inc.). FITC-labeled nanoparticles were coated with F(ab)2 portion of antibodies using known techniques. (See O'shannessy D J, Quarles R H., Labeling of the oligosaccharide moieties of immunoglobulins. J Immunol Methods. 1997;99: 153-61; Roberts J C, Adams Y E, Tomalia D, Mercer-Smith J A, Lavallee D K, Using starburst dendrimers as linker molecules to radiolabel antibodies. Bioconjug Chem. 1990; 1: 305-8; Sugano M, et al. Antibody Targeting of Doxorubicin-loaded Liposomes Suppresses the Growth and Metastatic Spread of Established Human Lung Tumor Xenografts in Severe Combined Immunodeficient Mice. Cancer Res. 2000;60: 6942-9; incorporation by reference herein.) For example, hydroxyl groups on HPC and HA particles are oxidized with pyridinium chlorochromate and then hydazide to form CONHNH2 group. The hydroxyl groups of the F(ab)2 are oxidized with sodium periodate to form an aldehyde group. Hydrazide (on HPC and HA particles) and amine (on NIPA particles) are then reacted with the aldehyde group on the F(ab)2 to form covalent bonds. Tagged, antibody-conjugated nanoparticles may be used after dialysis with sterile saline.

A series of NIPA nanoparticles with amine functional groups of different sizes (˜10 μm to 50 nm diameter) were also produced and then conjugated with a tag, such as fluorescein-isothiocyanate (FITC). These tagged and functionalized particles are able to cross one or more physiologic barriers as illustrated below.

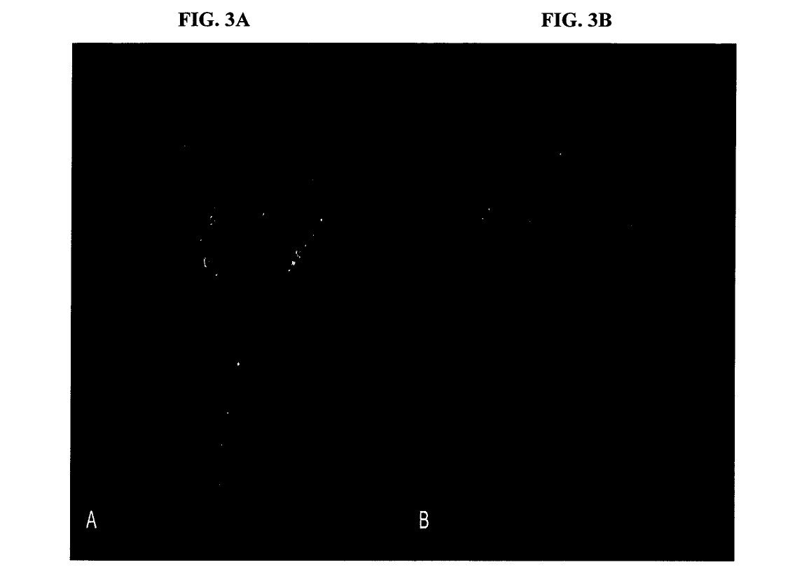

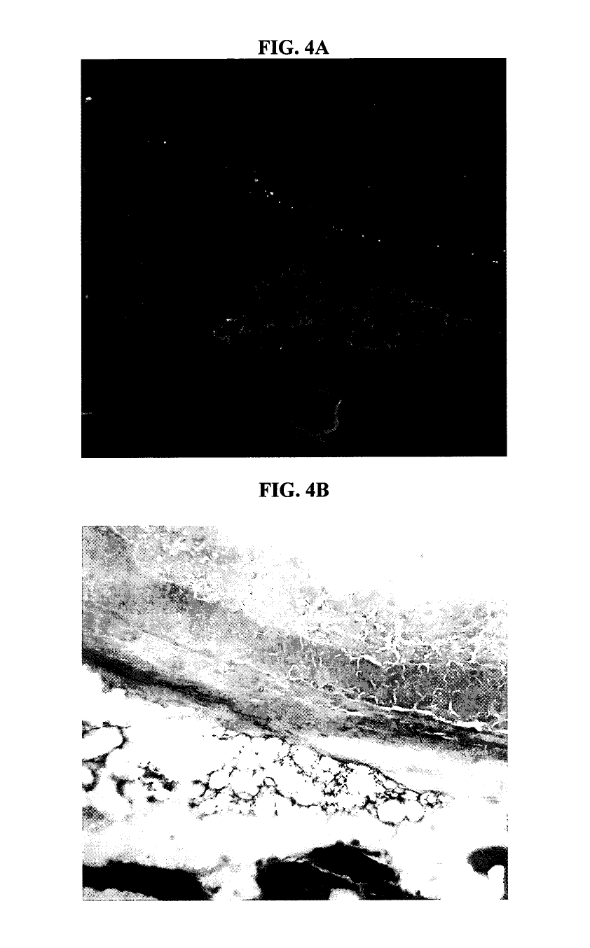

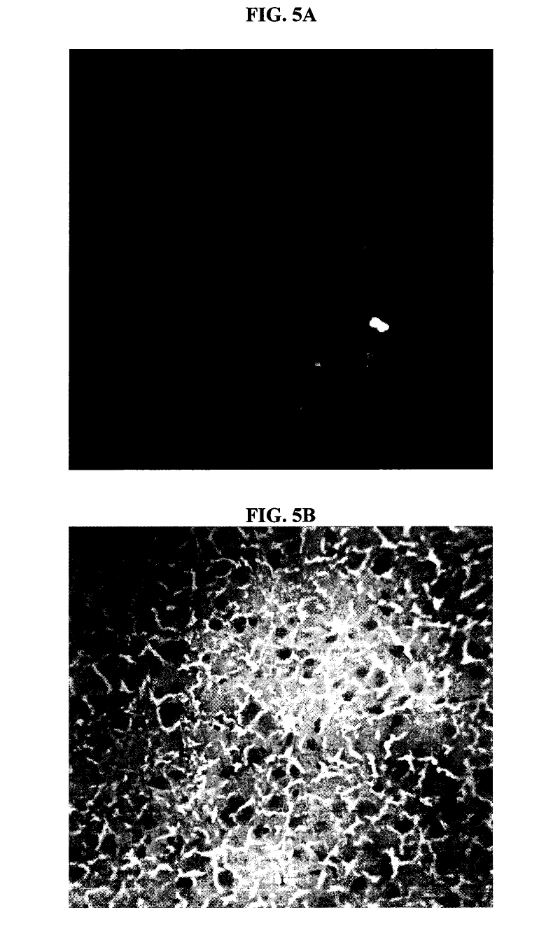

FIG. 3 shows the penetration and accumulation of intravenously administered NIPA nanoparticles (R<700 nm) in rat uveal tissue (see arrows), wherein one day after injection, eyes were recovered and then frozen sectioned and the distribution of FITC-labeled NIPA nanoparticles (dots) monitored using fluorescent microscopy. FIG. 3A shows FITC-labeled NIPA nanoparticles; FIG. 3B shows the saline injection control. The accumulation of intravenously injected NIPA nanoparticles (with amine groups) is depicted in FIG. 4A. These amine-rich nanoparticles remained in the uveal tissue for a prolonged period of time (>7 days). In addition, the amine-rich NIPA nanoparticles do not trigger any foreign body reactions in the uveal tissue as depicted using an H&E stain of the same tissue (FIG. 4B).

NIPA-tagged nanoparticles (˜100 nm in size) also cross the blood-brain barrier and penetrate as well as aggregated outside the vasculature in the brain stem at least seven days after intravenous administration of the functionalized particles (FIG. 5A). Importantly, the NIPA-tagged particles do not elicit any foreign body reactions in the brain stem (FIG. 5B).

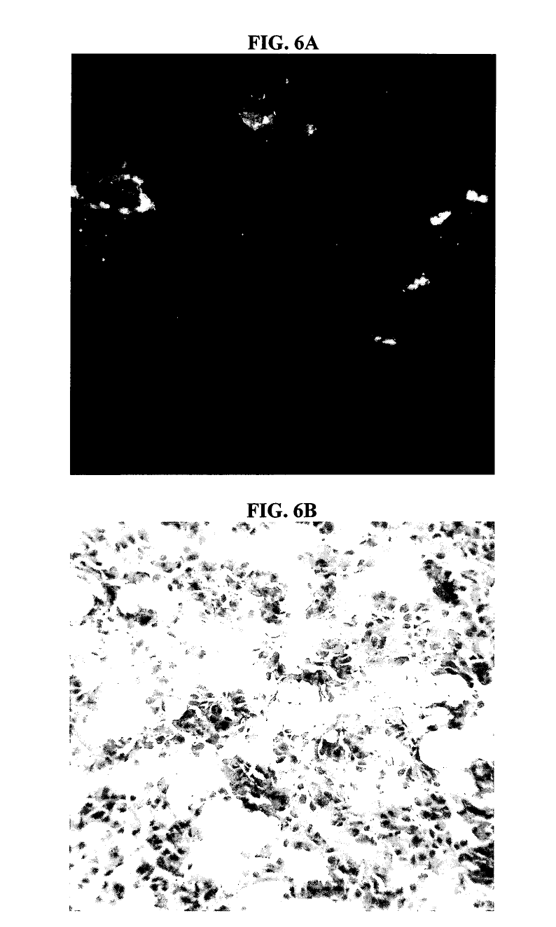

Similarly, tagged NIPA nanoparticles (˜100 nm in size) quickly cross the endothelial junction barrier and penetrate as well as accumulate outside the capillaries in lung tissue. The nanoparticles remain in the lung tissue for more than 7 days (FIG. 6A) and after examining lung tissue infiltrated with nanoparticles, no visible foreign body reactions were observed (FIG. 6B).

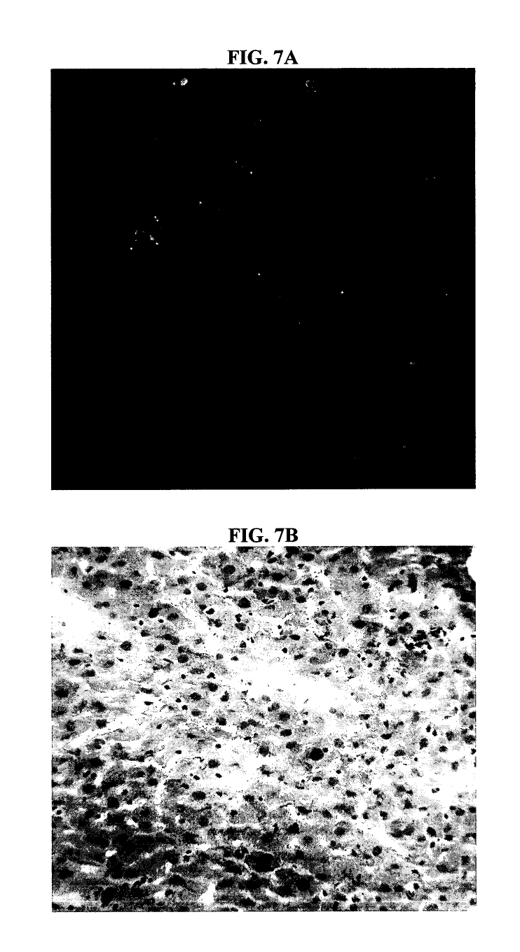

Liver, a highly vascularized organ, may also be targeted by nanoparticles of the present invention. For example, following intravenous injection with tagged NIPA nanoparticles (˜100 nm in size, tagged with FITC), nanoparticles are found to accumulate in the tissue, and remain, even after more than four days (FIG. 7A). These particles do not affect liver cell morphology nor do they prompt any foreign body reactions (FIG. 7B).

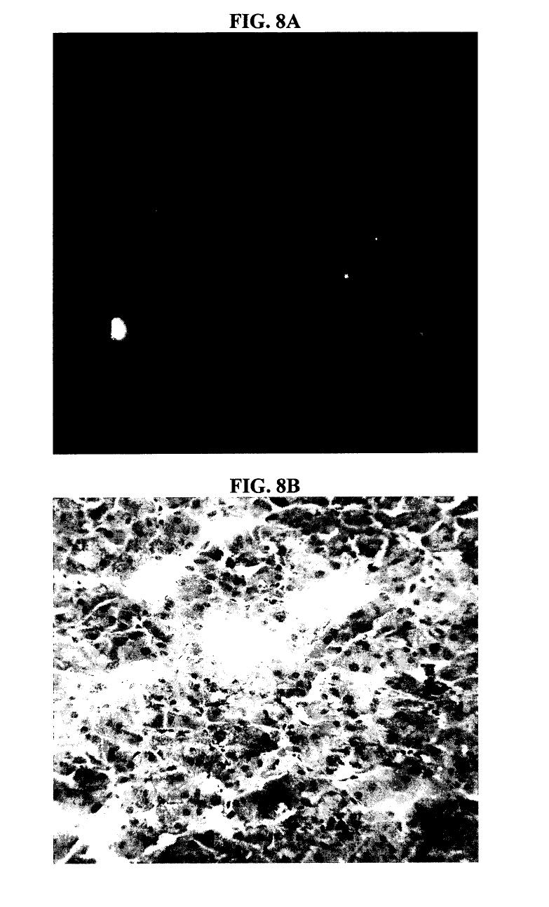

The pancreas may also be targeted by nanoparticles of the present invention. Following intravenous injection with tagged NIPA nanoparticles (˜100 nm in size, tagged with FITC), nanoparticles are found to accumulate in the tissue, and remain, even after more than four days (FIG. 8A). These particles do not affect pancreas cell morphology nor do they prompt any foreign body reactions (FIG. 8B).

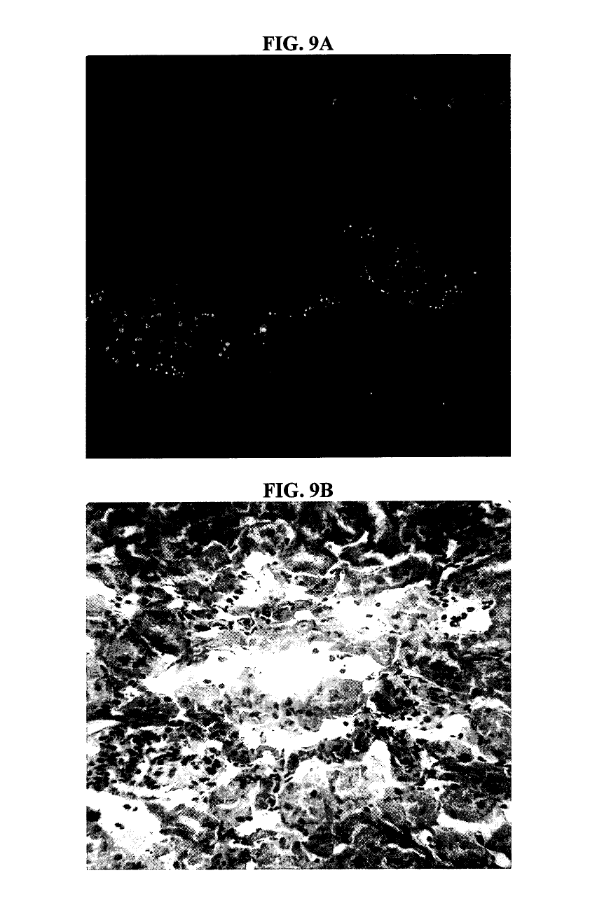

The kidney may also be targeted by nanoparticles of the present invention. Following intravenous injection with tagged NIPA nanoparticles (˜100 nm in size, tagged with FITC), nanoparticles are found to accumulate in the kidney, and remain, even after more than four days (FIG. 9A). These particles do not affect kidney cell morphology nor do they prompt any foreign body reactions (FIG. 9B).

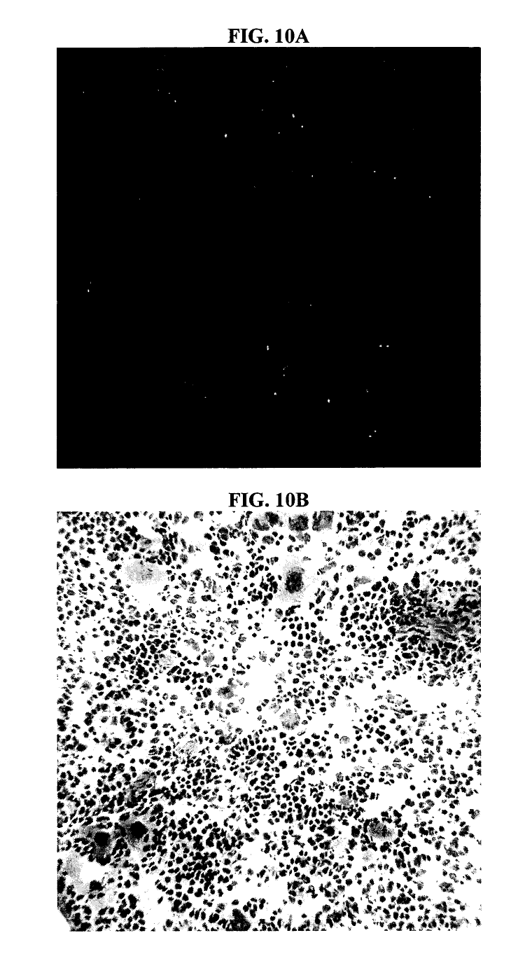

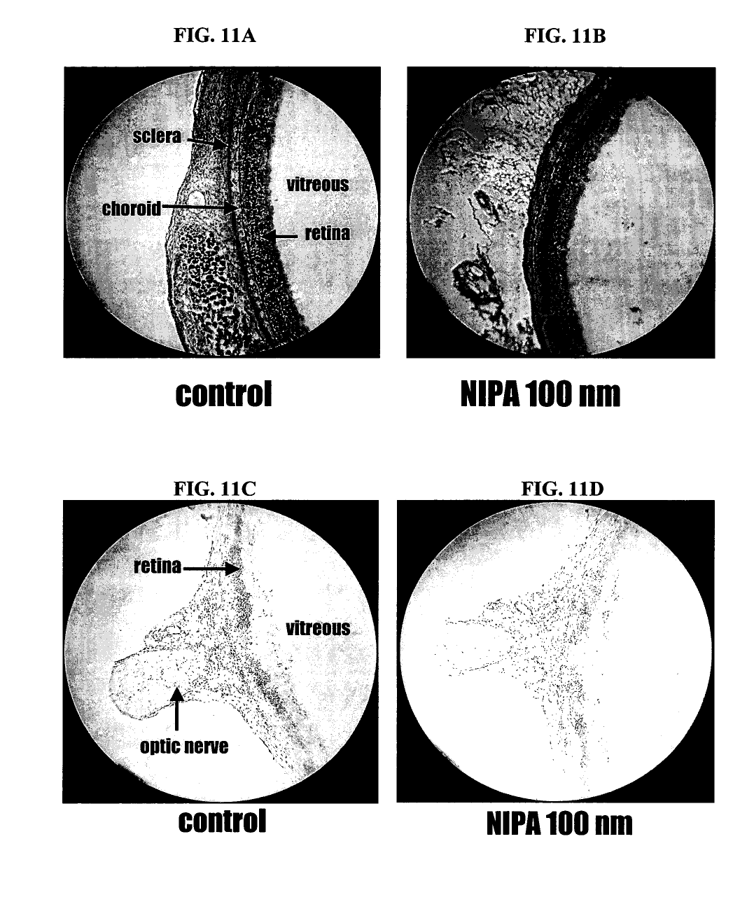

The spleen may also be targeted by nanoparticles of the present invention. Following intravenous injection with tagged NIPA nanoparticles (˜100 nm in size, tagged with FITC), nanoparticles are found to accumulate in the spleen, and remain, even after more than four days (FIG. 10A). These particles do not affect spleen cell morphology nor do they prompt any foreign body reactions (FIG. 10B).

The nanoparticles of the present invention may be delivered directly to specific cells, organs, tissue or tissue spaces. For example, intravitreal injection of tagged NIPA nanoparticles (˜100 nm, tagged with FITC) was performed. The nanoparticles did not lead to any adverse responses in the tissue (FIG. 11B) by comparison to control or untreated tissue (FIG. 11A). Upon implantation of the nanoparticles, there was no record of any inflammatory reaction (e.g., no accumulation of inflammatory cells) (FIG. 11D) in the retinal tissue by comparison to control or untreated tissue (FIG. 11C).

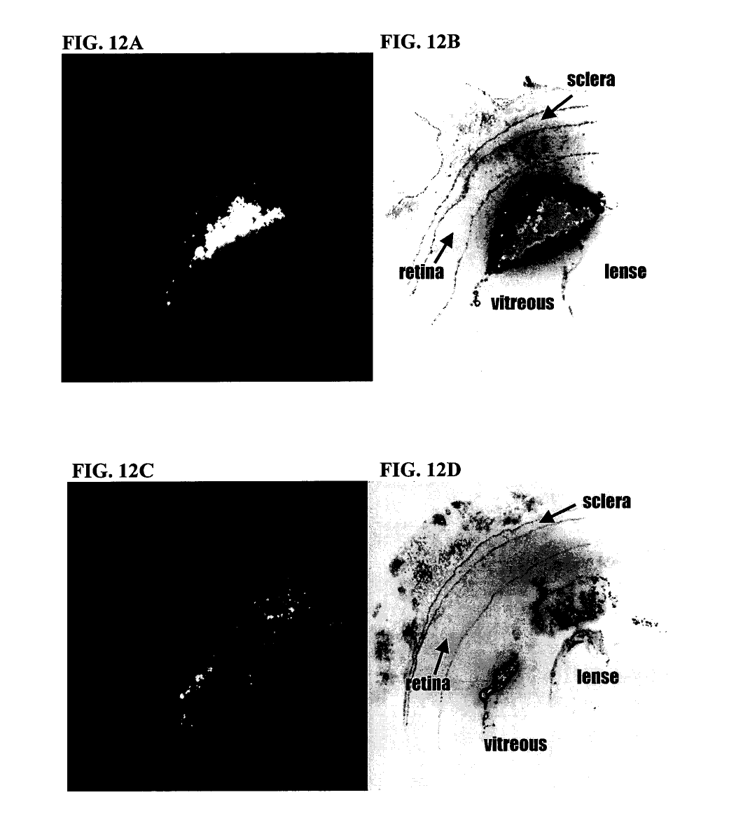

Following direct delivery of the nanoparticles of the present invention, the nanoparticles are found to migrate to other areas of that particular tissue. For example, following intravitreal injection of tagged NIPA microparticles (˜50 μm, tagged with FITC), the nanoparticles aggregated and accumulated in the intravitreal space, which is some distance from the site of injection (i.e., the retinal cells) (FIGS. 12A and 12B). Tagged nanoparticles of smaller size (˜100 nm, tagged with FITC) were found to aggregate and accumulate in the intravitreal space (FIGS. 12C and 12D).

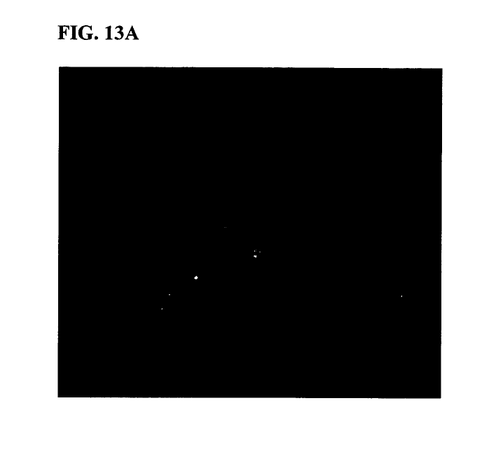

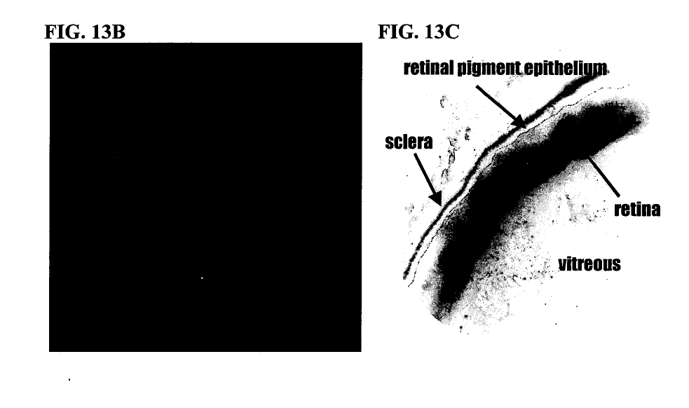

Affinity-enhanced particles of the present invention (those with functional modification such as amine modifications) are found to penetrate retinal tissue rapidly. Tagged NIPA-amine nanoparticles (˜100 nm, tagged with FITC) were observed 3 hours following implantation via intravitreal injection. The nanoparticles were found distribute evenly along the retina (FIG. 13A) and after a few hours, penetrated further into the tissue and remained in the retina for more than a week (FIG. 13B and C). Amine-rich (e.g., positively charged) functionalized particles using hyaluronic acid are also able to penetrate and target retinal tissue. (data not shown)

Similar observations have been made following delivery of functionalized and tagged nanoparticles to other tissues. Tissue delivery methods include intraocularly, intracranial, intrathecal, by injection, by inhalation, via an epidural, and to the joint. In all cases, nanoparticles were able to cross a physiologic barrier such as the endothelial cell junction and penetrate as well as accumulate into areas around and away from the site of delivery while still remaining within the tissue of origin. (data not shown) Improved tissue penetration and accumulation is achieved when using particles with diameter less than 700 nm. (data not shown) When the functionalized particle was tagged for a targeted tissue or cell, the functionalized particle would accumulate in the specific tissue for several days or weeks. (data not shown) Similar results may be obtained when functionalized particles are used as gene delivery vehicles to cross physiologic barriers such as the eye-blood or blood-brain barrier. Thus, animals, including humans, may be immunized by receiving a particle-antigen combination (encapsulated or not) in which the particles result in an increase in secretory and systemic antibodies in the blood.

To attach a drug to the functionalized particle of the present invention, one or more drugs are loaded (in acetone) and incorporated into the nano- or micro-particles of the present invention by adding a pre-determined volume of drug from a stock solution to the polymer solution and mixing to ensure uniform distribution. The drug-containing polymer solution is used to produce drug-tagged particles. Loading doses of the functionalized particles are based on the published therapeutic dosage for each drug. The amount of drug loaded per mg of polymer and the percent loading is determined by redissolving a known amount of the particles in acetone and then analyzing for the drug content by high-performance liquid chromatography (HPLC) assay using published measurement techniques. (See MacCallum J, et al., Solid-phase extraction and high-performance liquid chromatographic determination of tamoxifen and its major metabolites in plasma. J Chromatogr. 1996;678: 317-23; herein incorporated by reference.) Similar strategies may be developed to specifically target any cell, tissue or organ.

The present invention provides methods and compositions to safely, selectively, and effectively treat diseases, conditions, injuries, and abnormalities of the eye and other organs in a mammal.

In another embodiment, the present invention is a method for the administration of drugs affecting an organ or tissue to produce a physiologic or pharmacologic effect, or to apply substances with diagnostic value, that overcomes the physiologic barriers of the tissue or organ. In addition, the present invention is a method of treating a patient with an ocular disease with a drug-nanoparticle combination by administering said drug-nanoparticle to a patient in need thereof by mouth, intraperitoneally, topically, intranasally, intravenously, intraocular, intracranial, intrathecal, intramuscular, within the joint or as an epidural, and in amounts to provide an active dose.

In yet another embodiment, the present invention is a method of enhancing ocular drug delivery to the posterior portion of the eye by preparing an ocular drug comprising drug-nanoparticle combination, wherein the drug is for one or more ocular diseases selected from the group consisting of uveal disease, retinal disease, ocular tumor, and scleral disease; and introducing the drug-nanoparticle combination to a patient in need thereof. Still another embodiment is an ocular drug delivery system, comprising a nanoparticle for delivering an ocular drug to the eye, a drug associated with the nanoparticle, and a means for administration of the nanoparticle and drug into the body of patient in need thereof.

Yet another embodiment of the present invention is a method of transmitting an active drug across a physiologic barrier in a mammal to achieve an active dose by mixing a nanoparticle with an active drug, wherein the drug is selected from the group consisting of protective and therapeutic, and administering the nanoparticle with an active drug to the mammal, wherein the active drug is able to cross the physiologic barrier and exert a protective or therapeutic effect.

Still another embodiment is a method of treating uveal melanoma comprising the administration of a nanoparticles coated with an antibody, antibody fragments, peptide, other molecular ligand or combination thereof to a patient in need thereof. The monoclonal antibody may be selected from the group consisting of gp100, melan-A, vimentin, keratin, or specific cellular/tissue surface marker(s). Methods of administration include those well known in the art and in amounts to provide an active dose that is therapeutic.

As such, the present invention offers a number of advantages to current therapeutic and diagnostic methods and compositions in that the present invention may be administered systemically, but at a reduced therapuetically effective dose because it is targeted specifically to an organ and/or tissue. The small (e.g., nanoparticle) size allows particles of the present invention to cross physiologic barriers and effectively penetrate and accumulate in an organ or tissue and at doses that reduce peripheral side effects. Furthermore, by conjugating the functionalized particle with cell or tissue specific antigens, antibodies, molecular ligands, or peptide sequences, these modified and funcationalized particles can accumulate in specific organs for prolonged period of time to achieve improved therapeutic effects.

Additional objects, advantages and novel features of the invention as set forth in the description, will be apparent to one skilled in the art after reading the foregoing detailed description or may be learned by practice of the invention. The objects and advantages of the invention may be realized and attained by means of the instruments and combinations particularly pointed out here.

1. A method of delivering a particle to a mammal comprising the steps of:
contacting a functionalized particle with a tag; and
introducing the functionalized and tagged particle to a mammal,
wherein the functionalized portion of the particle is selected from the group consisting of acrylic acid, 2-hydroxyethyl acrylate, 2-acrylamido-2-methyl-1-propanesulfonic acid, allylamine, carboxyl group, hydroxyl group, sulfonic group, aldehyde group and amine group, and wherein the particle is a biodegradable or nodegradable polymer and less than 1.0 mm in diameter.

2. The method of claim 1, wherein the functionalized particle is a polymer selected from the group consisting of polyelectrolyte, hydroxypropyl cellulose, N-isopropylacrylamide, and hyaluronan.

3. The method of claim 1, wherein the tag is selected from the group consisting of drug, antibodiy, ligand, antigen, protein, peptide, nucleic acid sequence, fatty acid moiety, carbohydrate moiety, label, light-emitting species, radioactive species, nuclear species, contrast agent, and combinations thereof.

4. The method of claim 1, wherein the particle is protective, diagnostic, or therapeutic for one or more diseases selected from the group consisting of the eye, liver, brain, pancreas, spleen, kideny, and lung.

5. A method of using a functionalized particle to treat a patient in need thereof comprising the step of:
introducing the functionalized particle to the patient,
wherein the functionalized portion of the particle is selected from the group consisting of acrylic acid, 2-hydroxyethyl acrylate, 2-acrylamido-2-methyl-1-propanesulfonic acid, allylamine, carboxyl group, hydroxyl group, sulfonic group, aldehyde group and amine group, wherein the particle is a biodegradable or nodegradable polymer and less than 1.0 mm in diameter, and wherein the functionalized particle is introduced to the patient intraocularly, by injection, or by mouth.

6. The method of claim 5, wherein the functionalized particle is a polymer selected from the group consisting of polyelectrolyte, hydroxypropyl cellulose, N-isopropylacrylamide, and hyaluronan.

7. The method of claim 5, wherein the functionalized particle is further modified with a tag selected from the group consisting of drug, antibodiy, ligand, antigen, protein, peptide, nucleic acid sequence, fatty acid moiety, carbohydrate moiety, label, light-emitting species, radioactive species, nuclear species, contrast agent and combinations thereof.

8. The method of claim 5, wherein the functionalized particle is less than 700 nm.

9. A functionalized particle comprising:
a functionalized particle, wherein the functionalized portion of the particle is selected from the group consisting of acrylic acid, 2-hydroxyethyl acrylate, 2-acrylamido-2-methyl-1-propanesulfonic acid, allylamine, carboxyl group, hydroxyl group, sulfonic group, aldehyde group and amine group, and wherein the particle is a biodegradable or nodegradable polymer and less than 1.0 mm in diameter; and
a tag contacting the functionalized particle.

10. The functionalized particle of claim 9, wherein the tag is selected from the group consisting of drug, antibodiy, ligand, antigen, amino acid sequence, nucleic acid sequence, fatty acid moiety, carbohydrate moiety, label, light-emitting species, radioactive species, nuclear species, contrast agent, and combinations thereof.

11. The functionalized particle of claim 9, wherein the particle is for a patient in need thereof for diagnosis, prevention or treatment.

12. The functionalized particle of claim 9, wherein the functionalized particle is a polymer selected from the group consisting of polyelectrolyte, hydroxypropyl cellulose, N-isopropylacrylamide, and hyaluronan.

13. A method of enhancing delivery of a particle to the posterior portion of the eye, comprising the steps of:
preparing an ocular particle comprising a functionalized particle, wherein the functionalized portion of the particle is selected from the group consisting of acrylic acid, 2-hydroxyethyl acrylate, 2-acrylamido-2-methyl-1-propanesulfonic acid, allylamine, carboxyl group, hydroxyl group, sulfonic group, aldehyde group and amine group, and wherein the particle is a biodegradable or nodegradable polymer and less than 1.0 mm in diameter; and
introducing the ocular particle to a patient in need thereof.

14. The method of claim 13, wherein the ocular particle is introduced intraocularly, by injection, or by mouth.

15. The method of claim 13, wherein the ocular particle further comprises a tag selected from the group consisting of drug, antibodiy, ligand, antigen, protein, peptide, nucleic acid sequence, fatty acid moiety, carbohydrate moiety, label, light-emitting species, radioactive species, nuclear species, constrast agent and combinations thereof.

16. The method of claim 13, wherein the functionalized particle is a polymer selected from the group consisting of polyelectrolyte, hydroxypropyl cellulose, N-isopropylacrylamide, and hyaluronan.

17. A method of crossing a physiologic barrier with a functionalized particle comprising the steps of:
contacting the functionalized particle with a tag, wherein the functionalized portion of the particle is selected from the group consisting of acrylic acid, 2-hydroxyethyl acrylate, 2-acrylamido-2-methyl-1-propanesulfonic acid, allylamine, carboxyl group, hydroxyl group, sulfonic group, aldehyde group and amine group, and wherein the particle is a biodegradable or nodegradable polymer and less than 1.0 mm in diameter; and
administering the functionalized particle to a mammal, wherein the functionalized particle is capable of crossing the physiologic barrier and exerts an effect.

18. The method of claim 17, wherein the tag is selected from the group consisting of drug, antibodiy, ligand, antigen, protein, peptide, nucleic acid sequence, fatty acid moiety, carbohydrate moiety, label, light-emitting species, radioactive species, nuclear species, contrast agent, and combinations thereof.

19. The method of claim 17, wherein the functionalized particle is a polymer selected from the group consisting of polyelectrolyte, hydroxypropyl cellulose, N-isopropylacrylamide, and hyaluronan.

20. The method of claim 17, wherein the effect is selected from the group consisting of diagnostic, therapeutic, protective and preventative.

21. The method of claim 17, wherein administering is selected from the group consisting of intraocularly, by injection, or by mouth.

22. The method of claim 17, wherein the functionalized particle is less than 700 nm.

23. A method of crossing a physiologic barrier with a functionalized particle comprising the steps of:
preparing a functionalized N-isopropylacrylamide particle with a tag, wherein the functionalized portion of the particle is selected from the group consisting of acrylic acid, 2-hydroxyethyl acrylate, 2-acrylamido-2-methyl-1-propanesulfonic acid, allylamine, carboxyl group, hydroxyl group, sulfonic group, aldehyde goup, and amine group, and wherein the particle is less than 1.0 mm in diameter; and
administering the functionalized N-isopropylacrylamide particle, wherein the functionalized N-isopropylacrylamide particle is capable of crossing the physiologic barrier and exerts an effect.

24. The method of claim 23, wherein the tag is selected from the group consisting of drug, antibodiy, ligand, antigen, protein, peptide, nucleic acid sequence, fatty acid moiety, carbohydrate moiety, label, light-emitting species, radioactive species, nuclear species, contrast agent, and combinations thereof.

25. The method of claim 23, wherein the effect is selected from the group consisting of diagnostic, therapeutic, protective and preventative.

26. The method of claim 23, wherein administering is selected from the group consisting of intraocularly, by injection, or by mouth.

27. The method of claim 23, wherein the functionalized N-isopropylacrylamide particle is less than 700 nm.
